SEQUENCE LISTING (2 sequences)

SEQ ID NO: 1 - DNA, 2139 bp, Artificial Sequence
  DNA sequence for encoding isoamylase derived      from Acidothermus cellulolyticus ATCC 43068
atgccggaaccgacgacgcaatccctcgaggtctggccgggcgatccgtatccgctcggtgccacgtatgacggggcgggcacgaacttcgccgttttctccgaggtcgccgagcagatcgagctctgcctcttcgacgacgacgggaatgagactcgggtcaccctcccggagtacgacgcattcgtctggcacggctacctgccggggatcggcccggggacacggtacgggttccgcgtgcacggcccctatgaccccgcccgcggtctgcggtgcaatccggcgaaactcctcctcgacccatacgccaaagccatcgacggggacattgacggtcacgaatcgctcttcggttaccgattcggcgatccggcgagccgcaacgatgaagactccgcgccgcacatgatgaagtcggttgtcatcaatccgttcttcgactggcgcaatgaccatccgctccgcacgccgtaccacgagtcgatcatttacgaggcgcacgtgcgcggcatgacgatgacgcacccggagattcccgagcatttgcgcggtacgtacgccgcgctcgcgcacccggtgatgctcgactacctgtcttccctgggagtgacggccgtcgagctgatgccggtgcatcaattcgtcaccgataccgcgctgcgggagcgcggcctgcgcaattactggggatacaacacgatcggcttctttgccccgcacaacggatattccgcatccggcagtagaggcgagcaggtgcaggagttcaaggccatggtccgcgccctgcatgaagccggtatcgaggtgatcctggacgtcgtctataaccacactgctgaaggcaatcacctcgggcctaccctctcattccgcggcctggacaacgcggcgtattaccgcctggcggatgacgatcccagccggtacgtcgactacaccggcaccggtaacagtttcaacgcccgcaatccgcatgccttgcagctcatcatggattccctccgttactggattctcgagatgcacgtcgacgggttccggttcgaccttgcgtccgcgttggcccgtgaattgcacgacgtcgaccggctctcggccttcttcgacctcgtacagcaggatccggtggtgagccaggtcaagctcatcgccgagccgtgggacgtcggcgagggcggttaccaggtcggcaatttcccgccgctgtggagcgagtggaacggtaaataccgcgacacggtgcgggatttctggcgcggtgagccggcgactctcggtgaattcgcctcccggctgaccggctcctcggacctgtacgcgagctcgggccgccgtccgatggcgtccatcaacttcgtcacctgccacgacggcttcaccttgcacgacctggtctcgtacaacgagaagcacaacgaggccaacggtgagggaaaccgtgacggcagcgacgacaatcggtcatggaactgcggtgtcgaaggacccacggacgacgtgcacatcatcgcgcttcgggagcaacaaaagcgcaatttcctcacgacgctcctgctctcgcagggcgttcccatgattctgcacggggacgagttcgggcgaacccaacggggcaacaacaacgcctactgccaggacaacgaaatctcctggatggattggcggctcgcagtcgagcacgaggtgcaactcagcttcacccgaaagctcaccacgttccggaaagaacacccggtcttccgccggcggcggttcttcgacggcaagccggttccgcatgtcgccggcgaggcactgccggatatcgcctggttcaccccggccgcggcgctgatgaccgagacggactgggagaccgggtatgccaagagcctgaccgtcttcgtcaacggcgatgcgatcccgtcacccgaccgccgcggccagccggtgcgggacgattccttcctgttgctgttcaacgccgacgcgaacgacctcgaattccgccttcccgacgaggagtacggccagcgatgggaggccgtcatcgacaccaccgatcccctgctcatcgatcccccgacgtacaaggcacaggcggcggtcaccgtgccggctcgatgcgttctggtgctgcgccgtgtcctctaa

SEQ ID NO: 2 - protein, 712 aa, Artificial Sequence
  Isoamylase derived from Acidothermus      cellulolyticus ATCC 43068
MPEPTTQSLEVWPGDPYPLGATYDGAGTNFAVFSEVAEQIELCLFDDDGNETRVTLPEYDAFVWHGYLPGIGPGTRYGFRVHGPYDPARGLRCNPAKLLLDPYAKAIDGDIDGHESLFGYRFGDPASRNDEDSAPHMMKSVVINPFFDWRNDHPLRTPYHESIIYEAHVRGMTMTHPEIPEHLRGTYAALAHPVMLDYLSSLGVTAVELMPVHQFVTDTALRERGLRNYWGYNTIGFFAPHNGYSASGSRGEQVQEFKAMVRALHEAGIEVILDVVYNHTAEGNHLGPTLSFRGLDNAAYYRLADDDPSRYVDYTGTGNSFNARNPHALQLIMDSLRYWILEMHVDGFRFDLASALARELHDVDRLSAFFDLVQQDPVVSQVKLIAEPWDVGEGGYQVGNFPPLWSEWNGKYRDTVRDFWRGEPATLGEFASRLTGSSDLYASSGRRPMASINFVTCHDGFTLHDLVSYNEKHNEANGEGNRDGSDDNRSWNCGVEGPTDDVHIIALREQQKRNFLTTLLLSQGVPMILHGDEFGRTQRGNNNAYCQDNEISWMDWRLAVEHEVQLSFTRKLTTFRKEHPVFRRRRFFDGKPVPHVAGEALPDIAWFTPAAALMTETDWETGYAKSLTVFVNGDAIPSPDRRGQPVRDDSFLLLFNADANDLEFRLPDEEYGQRWEAVIDTTDPLLIDPPTYKAQAAVTVPARCVLVLRRVL

(19) United States
(12) Patent Application Publication
(10) Pub. No.: US 2019/0136277 A1
(43) Pub. Date: May 9, 2019

(54) METHOD FOR PREPARING GLUCOSE FROM STARCH SUGAR BY USING ACIDOTHERMUS SP.-DERIVED DEBRANCHING ENZYME HAVING HEAT RESISTANCE AND ACID RESISTANCE, AND GLUCOSE PREPARED THEREBY

(71) Applicant: CJ CHEILJEDANG CORPORATION, Seoul (KR)

(72) Inventors: Jungap AN, Suwon-si (KR); Ran Young YOON, Suwon-si (KR); Seong Bo KIM, Seongnam-si (KR); Seung Won PARK, Yongin-si (KR)

(73) Assignee: CJ Cheiljedang Corporation, Seoul (KR)

(21) Appl. No.: 16/067,478

(22) PCT Filed: Jan. 2, 2017
(86) PCT No.: PCT/KR2017/000030

§ 371 Date: Jun. 29, 2018

(30) Foreign Application Priority Data
Dec. 31, 2015 (KR) 10-2015-0191033

(51) Int. Cl. C12P 19/02

(52) U.S. Cl. CPC C12P 19/02; C08L 3/02

(57) ABSTRACT

The present application relates to improving the production yield of glucose to be prepared by using starch sugar as a raw material and using an Acidothermus sp. derived enzyme. The present application can replace an acid saccharification method, which produces a bitter taste through a hydrolysis reverse reaction, and, compared to a enzymatic saccharification method in which a conventional debranching enzyme is used, has an advantage of enabling costs to be reduced since a relatively high yield can be ensured by reducing side reactions of a saccharification reaction.

### TECHNICAL FIELD

The present invention relates to a method for producing glucose from starch sugars using a debranching enzyme derived from an Acidothermus sp. and a diastatic enzyme.

### BACKGROUND ART

Biomass can be broadly divided into sugar biomass (sugarcane), starchy biomass (corn and sweet potato), and cellulosic biomass (wood and silver grass) according to major ingredients. Sugars are easily obtained from sugarcane and can be directly ingested by organisms, avoiding the need for additional pretreatment or glycosylation. However, starchy biomass such as corn biomass is not directly available to microorganisms due to the high molecular weight of starch. For this reason, starch needs to be broken down into glucose. This course requires water and is thus called “hydrolysis”.

Starch is hydrolyzed mainly by enzymes, just as we digest rice. Cooked rice we eat is degraded into glucose in the stomach by enzymes secreted from salivary glands, and the glucose is transferred to body cells and is then used for metabolic processes. The first process for converting starch to glucose is to make the structure of starch loose, just as we cook rice by heating, with the result that enzymes can approach the starch and break it down to glucose. This enzymatic degradation process consists of inherently complex mechanisms.

An enzymatic glycosylation is a process for converting starch to glucose. According to this process, starch is liquefied by an alpha-amylase derived from a microorganism and the liquefied starch reacts with a debranching enzyme and a diastatic enzyme to produce glucose.

### DISCLOSURE

### Technical Problem

Promozyme® (Novozyme) is a pullulanase isolated from a Pseudomonas sp. and has been mainly used as a debranching enzyme for enzymatic glycosylation. However, due to its specificity for starch sugars with a narrow range of degrees of polymerization (DP 4-5), Promozyme® is unsuitable for the hydrolysis of branched chains of various lengths (DP 3-30) present in starch sugars, resulting in low glucose yield.

### Technical Solution

The present invention relates to a method for producing glucose from starch sugars using a novel debranching enzyme, and specifically, a method for producing glucose, which includes simultaneously adding a debranching enzyme derived from a heat-resistant and acid-tolerant Acidothermus sp. and a diastatic enzyme to a substrate solution containing one or more starch sugars selected from the group consisting of starch, amylopectin, and maltodextrin to react the enzymes with the starch sugars.

More specifically, starch sugars are treated with an isoamylase as a debranching enzyme derived from an Acidothermus sp. to hydrolyze branched chains of various lengths present therein, enabling the production of glucose from the starch sugars in high yield.

It is another object of the present invention to provide a method for producing glucose from starch sugars in high yield using an isoamylase derived from an Acidothermus sp. and a diastatic enzyme in a predetermined unit ratio.

In order to achieve the above and other objects of the present invention, one aspect of the present invention provides a method for producing glucose from starch sugars, including treating a substrate solution containing starch sugars with a debranching enzyme derived from an Acidothermus sp. and a diastatic enzyme.

Specifically, the debranching enzyme derived from an Acidothermus sp. may be produced by a method including inserting a gene encoding the debranching enzyme into a vector to produce an expression vector, transforming the expression vector into cells, and culturing the transformed cells. The method may further include centrifuging and/or disrupting the resulting culture. The culture can be disrupted by any suitable technique known in the art, for example, using an ultrasonic homogenizer.

The vector may be pET-28a(+).

The debranching enzyme derived from an Acidothermus sp. may be an isoamylase. The debranching enzyme has hydrolytic activity on α-1,6-bonds, particularly specificity for starch sugars with a wide range of degrees of polymerization(DP3-30), resulting in an increase in glucose yield.

The diastatic enzyme is an enzyme that can catalyze the hydrolysis of debranched starch sugars or starch sugars into glucose. An addition of the diastatic enzyme enables the final production of glucose from the chains of the starch sugars cleaved by the debranching enzyme. For example, the diastatic enzyme may be selected from a-amylases, (3-amylases, and glucoamylases. Specifically, the diastatic enzyme may be a glucoamylase. The diastatic enzyme catalyzes glycosylation in the pH range of 3 to 7, more specifically 3 to 6. The reaction temperature of the diastatic enzyme may be from 40° C. to 70° C., specifically from 55° C. to 65° C.

The Acidothermus sp. may be Acidothermus cellulolyticus strain. For example, the Acidothermus cellulolyticus strain may be Acidothermus cellulolyticus strain 11B [ATCC® 43068™]. The said strain-derived isoamylase is encoded by the sequence set forth in SEQ ID NO. 1 and has the amino acid sequence set forth in SEQ ID NO. 2.

The cells which can be transformed with the expression vector may be, for example, any one of E. coli, a microorganism belonging to the genus Bacillus, yeast (e.g., Baker's yeast), a microorganism belonging to the genus Corynebacterium and a plant.

The starch sugars may be any one or more selected from the group consisting of for example starch, amylopectin, and maltodextrin. Specifically, the starch sugar may be amylopectin-type starch. Amylopectin and maltodextrin may be prepared by adding an alpha-amylase to starch.

The starch sugars may be derived from any one crop selected from for example, corn, tapioca, potato, and rice, but may not be limited to that.

The starch sugars may be liquefied starch ones.

The substrate solution may be treated with the debranching enzyme derived from an Acidothermus sp. and the diastatic enzyme at a pH of 2 to 6, more preferably 3 to 5, most preferably 4.3.

The substrate solution may be treated with the debranching enzyme derived from an Acidothermus sp. and the diastatic enzyme at a temperature of 40° C. to 70° C., more preferably 50° C. to 65° C., most preferably 65° C.

The unit ratio of the debranching enzyme activity derived from an Acidothermus sp. to the diastatic enzyme activity may be the range from 2:1 to 60:1, specifically from 10:1 to 60:1, more specifically from 10:1 to 40:1.

The aid of the debranching enzyme is required to exceed the reaction equilibrium of the diastatic enzyme. The glucose yield increases in proportion to the unit ratio of enzyme activity. However, if the unit ratio of the diastatic enzyme activity to the debranching enzyme activity derived from an Acidothermus sp. exceed to 1:60, the glucose yield may be decreased. The diastatic enzyme plays a major role in the degradation of starch sugars to glucose (i.e. glycosylation) and the debranching enzyme plays an auxiliary role in glycosylation. Therefore, if the unit ratio of the debranching enzyme activity derived from an Acidothermus sp. to the diastatic enzyme activity is less than 2:1, the role of the diastatic enzyme in the degradation of starch sugars cannot be expected, resulting in a low glucose yield.

Another aspect of the present invention provides a method of using a debranching enzyme derived from an Acidothermus sp. in the production of glucose from starch sugars.

A further aspect of the present invention provides a debranching enzyme derived from Acidothermus cellulolyticus that is active at a temperature of 40° C. to 70° C. and a pH of 2 to 6. The debranching enzyme has a hydrolytic activity on a-1,6-bonds and good selectivity for chain lengths, particularly chain lengths corresponding to DPs of 3 and 4, 3 or more, or 4 or more, resulting in an increase in a glucose yield.

The debranching enzyme is active at a temperature of 40° C. to 70° C., more preferably 50° C. to 65° C., most preferably 60° C.

The debranching enzyme is active at a pH of 2 to 6, more preferably pH 3 to 5, most preferably pH 4.3.

### Advantageous Effects

According to the present invention, the use of the debranching enzyme (isoamylase) derived from an Acidothermus sp. enables the production of glucose from starch sugars in high yield compared to the use of a conventional commercial debranching enzyme (pullulanase).

In addition, the ratio between the diastatic enzyme and the debranching enzyme used in the multiple enzymatic reactions is adjusted such that the amounts of by-products(DP2) and unreacted products (DP3, DP4+) in the hydrolysis of starch sugars are reduced, resulting in an improvement in the yield of glucose from the starch sugars.

Particularly, the debranching enzyme used in the method for producing glucose from starch sugars according to the present invention maintains its activity under strong acid and/or high temperature conditions, thus greatly improving the yield of glucose.

### DESCRIPTION OF DRAWINGS

FIG. 1 shows several hydrolases and pathways for the degradation of starch sugar.

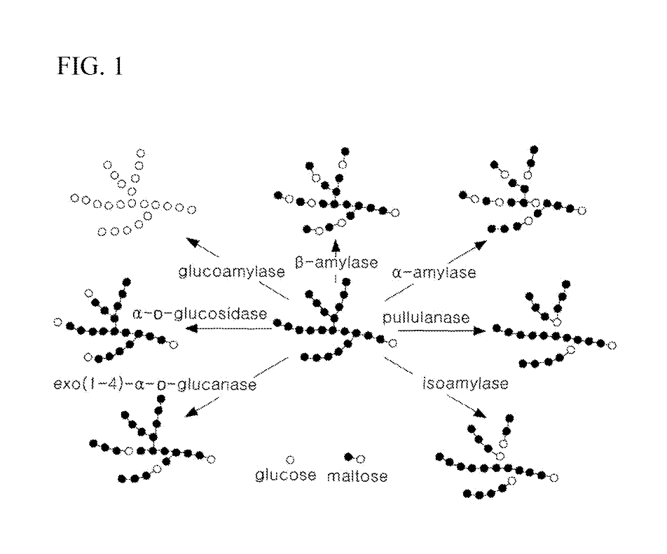

FIG. 2 shows several debranching enzymes and pathways for the degradation of starch sugar.

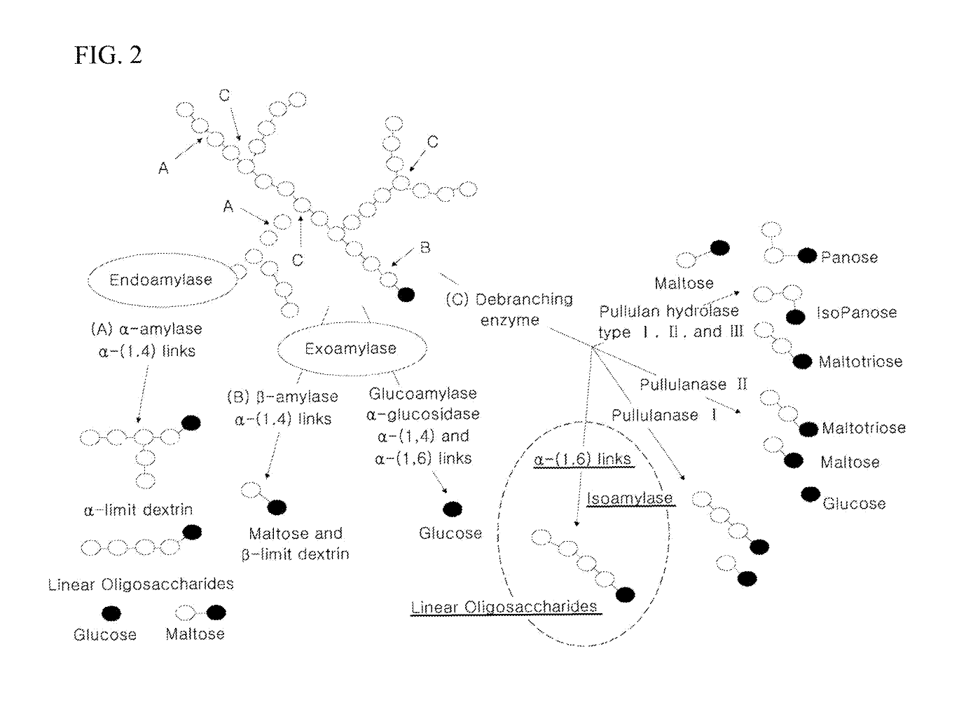

### MODE FOR INVENTION

The present invention will be described in more detail with reference to the following examples. These examples are merely illustrative to assist in understanding the invention and should not be construed as limiting the scope of the invention.

### EXAMPLE 1

### Gene Isolation and Recombinant Protein Production

1) Production of Recombinant E. coli

Forward and reverse primer sequences including portions of both end sequences of a debranching enzyme gene derived from a heat-resistant and acid-tolerant Acidothermus cellulolyticus and the recognition sequences of restriction enzymes NdeI and HindIII, respectively, were used for PCR.

(Forward) 5′-GAA-TTC-ATG-CCG-GAA-3′
(Reverse) 5′-GAA-TTC-TTA-GAG-GAC-3′

As a result, a PCR product of 2.1 kb was obtained. The acquired DNA fragment was inserted into pET-28a(+) vector and transfected into E. coli BL21(DE3). The DNA has the sequence set forth in SEQ ID NO. 1. The transformed E. coli was spread on the plate media containing kanamycin. Kanamycin-resistant strains were primarily screened and separately cultured in liquid media. DNA of Kanamycin-resistant strain was purified and digested with both NdeI and HindIII. The finally selected strain was found to have 2.1 kb and 5.3 kb DNA fragments. The sequence was analyzed using an automatic sequencer. As a result, the sequence was found to be identical to the gene sequence of the desired debranching enzyme.

2) Recombinant Protein Production

A debranching enzyme (isoamylase) was derived from acid-tolerant and heat-resistant Acidothermus cellulolyticus by the following procedure.

A test tube containing 5 ml of LB liquid medium was inoculated with frozen-stored recombinant E. coli strain BL21(DE3). The inoculum was cultured in an incubator at 37° C. until an absorbance of 2.0 at 600 nm was reached. The culture broth was added to 500 ml of LB liquid medium in a 2 L flask, followed by main culture. When the absorbance of the culture at 600 nm reached 0.4, 0.1 mM isopropyl β-D-1-thiogalactopyranoside (IPTG) was added to induce mass expression of a debranching enzyme. The temperature was maintained at 37° C. with stirring at 180 rpm during culture. Even after the addition of the IPTG, the culture was continued at 20° C. with stirring at 120 rpm. The culture broth of the transformed strain was centrifuged at 6,000×g and 4° C. for 20 min and washed twice with 50 mM Tris-Cl buffer. Then, Ni-NTA binding buffer (50 mM sodium phosphate, 300 mM NaCl, 10 mM imidazole, pH 8.0) was added and the cell solution was disrupted using an ultrasonic homogenizer. The cell lysate was centrifuged at 13,000×g and 4° C. for 20 min. The supernatant was collected as an enzyme extract and purified using a Ni-NTA superflow column to accurately characterize the enzyme. The purified enzyme was found to have a molecular weight of about-75 kDA, as determined by SDS-PAGE.

### EXAMPLE 2

### The Measurement of An-Enzymatic Activity by HPLC

1) The Activity of the Debranching Enzyme (Isoamylase) Derived from the Acid-Tolerant and Heat-Resistant Microorganism (Acidothermus cellulolyticus) was Measured by HPLC under the Following Conditions.

HPLC analysis conditions:
Detector: RID
Flow rate: 0.6 ml/min
Sample injection vol.: 10 μl
Column: Bio-Rad HPX 87C
Solvent: D.I. Water

The total time for analysis was set to 20 min.

The activity of the debranching enzyme (isoamylase) derived from the acid-tolerant and heat-resistant microorganism (Acidothermus cellulolyticus) was confirmed by the production of glucose from corn starch as a raw material through multiple enzymatic reactions with a diastatic enzyme (Glucoamylase). The enzymatic reaction conditions were as follows.

First, a diastatic enzyme (Glucoamylase, DuPont company) and the debranching enzyme (isoamylase, 50 mM sodium acetate buffer, pH 4.3) produced in Example 1 were used in a total unit ratio of 1:4. Multiple enzymatic reactions with liquefied starch having a dextrose equivalent (DE) of 9.5 were performed at a temperature of 60° C. for 72 h. The reactions were quenched by heating at 100° C. for 5 min. Then, the amount of glucose produced was measured by HPLC. The results of HPLC analysis revealed that the yield of glucose from the corn starch was 96.2%. The glucose yield was calculated from the ratio between the areas of peaks corresponding to the enzymatic reaction products.

### EXAMPLE 3

### The comparison of yields of glucose produced using the debranching enzyme (isoamylase) derived from Acidothermus sp. and commercial debranching enzyme (Pullulanase)

The yields of glucose from starch using the debranching enzyme derived from Acidothermus cellulolyticus and a commercial debranching enzyme (Pullulanase, Novozyme company) were investigated. The enzymatic reaction conditions were as follows.

First, a diastatic enzyme (Glucoamylase, DuPont company) and each of the debranching enzymes (isoamylase and pullulanase) were used in a total unit ratio of 1:4. Multiple enzymatic reactions with liquefied starch (DE 9.5) were performed at a temperature of 60° C. for 72 h. The reactions were quenched by heating at 100° C. for 5 min. Then, the amounts of glucose produced were measured from the ratios between the areas of peaks corresponding to the enzymatic reaction products by HPLC. As a result, the reactions using the debranching enzyme (isoamylase) derived from Acidothermus cellulolyticus gave a higher glucose yield than the reactions using a commercial debranching enzyme (Pullulanase). Starch sugars, including di-(DP2) and higher saccharides, were analyzed. As a result, when the debranching enzyme (isoamylase) derived from Acidothermus cellulolyticus was used, the proportions of the unreacted products (DP3, DP4+) were reduced, indicating relatively high yield of glucose.

TABLE 1

| Diastatic enzyme | Debranching enzyme | Glucose (%) | DP2 (%) | DP3 (%) | DP4+ (%) |
|---|---|---|---|---|---|
| Glucoamylase | Isoamylase derived from | 96.20 | 2.57 | 0.39 | 0.84 |
| (DuPont company) | Acidothermus sp. (Ratio 4) | | | | |
| (Ratio 1) | | | | | |
| | Pullulanase (Novozym | 96.10 | 2.16 | 0.64 | 1.10 |
| | company)(Ratio 4) | | | | |

As shown in Table 1, the use of the isoamylase derived from Acidothermus cellulolyticus in the present application led to a higher glucose yield due to its higher activity than the use of the pullulanase known to be optimal for mass production. The proportions of the unreacted products (DP3 and DP4+) with relatively high degrees of polymerization were smaller when the said isoamylase was used than when the pullulanase was used. From these results, it can be concluded that the application of the inventive isoamylase to enzymatic reactions leads to an increase in glucose yield.

### EXAMPLE 4

### The Changes in Glucose Yield with varying Concentrations of the Debranching Enzyme (Isoamylase) Derived from Acidothermus sp.

The changes in glucose yield with varying concentrations of the debranching enzyme (isoamylase) derived from Acidothermus cellulolyticus were investigated. The enzymatic reaction conditions were as follows. Multiple enzymatic reactions with liquefied starch (DE 9.5) were performed at a temperature of 60° C. for 72 h, wherein a diastatic enzyme (Glucoamylase, DuPont company) and the debranching enzyme were used in total unit ratios of 1:2, 1:10, 1:20, and 1:60, respectively. The reactions were quenched by heating at 100° C. for 5 min. Then, the amounts of glucose produced were measured from the ratios between the areas of peaks corresponding to the enzymatic reaction products by HPLC. As a result, the higher the ratio of the debranching enzyme (isoamylase) to the diastatic enzyme, the higher the glucose yield. These results appear to be because the higher activity of the debranching enzyme (isoamylase) derived from Acidothermus cellulolyticus leads to the production of smaller amounts of by-products (DP2) formed in the starch hydrolysis and the degradation of larger amounts of the unreacted products (DP4) by the diastatic enzyme, resulting in an increase in glucose yield.

TABLE 2

| Diastatic enzyme | Debranching enzyme | | | | |
|---|---|---|---|---|---|
| (glucoamylase, DuPont | (Isoamylase from | | | | |
| company) | Acidothermus) | Glucose (%) | DP2 (%) | DP3 (%) | DP4+ (%) |
| Ratio 1 | Ratio 2 | 95.91 | 2.31 | 0.57 | 1.21 |
| | Ratio 10 | 96.71 | 2.15 | 0.60 | 0.54 |
| | Ratio 20 | 96.94 | 1.99 | 0.63 | 0.43 |
| | Ratio 60 | 97.07 | 1.90 | 0.63 | 0.40 |

As shown in Table 2, glucose was produced in high yield with the increasing total unit ratio of the diastatic enzyme to the isoamylase from 1:2 to 1:60. That is, the yield of glucose increased with the increasing ratio of the isoamylase to the diastatic enzyme. cl EXAMPLE 5

### The Changes in Glucose Yield with the Varying Concentrations of Diastatic Enzyme in Multiple Reactions of the Debranching Enzyme Derived from an Acidothermus sp. and the Diastatic Enzyme

The changes in glucose yield with the varying concentrations of a diastatic enzyme (Glucoamylase, DuPont company) in multiple reactions of the debranching enzyme (isoamylase) and the diastatic enzyme were investigated. The enzymatic reaction conditions were as follows. Multiple enzymatic reactions with liquefied starch (DE 9.5) were performed at a temperature of 60° C. for 72 h, wherein the debranching enzyme and the diastatic enzyme were used in total unit ratios of 1:0.05, 1:0.04, 1:0.03, and 1:0.02, respectively. The reactions were quenched by heating at 100° C. for 5 min. Then, the amounts of glucose produced were measured from the ratios between the areas of peaks corresponding to the enzymatic reaction products by HPLC. As a result, the smaller the proportion of the diastatic enzyme, the higher the glucose yield. When the diastatic enzyme (glucoamylase) in the reaction for starch hydrolysis reached the equilibrium of hydrolysis, by-products (DP2) were formed. This problem was solved by lowering the concentration of the diastatic enzyme such that the formation of by-products is reduced, resulting in an increase in the relative yield of glucose.

TABLE 3

| Debranching enzyme | Diastatic enzyme | | | | |
|---|---|---|---|---|---|
| (Isoamylase from | (Glucoamylase, DuPont | | | | |
| Acidothermus) | company) | Glucose (%) | DP2 (%) | DP3 (%) | DP4+ (%) |
| Ratio 1 | Ratio 0.05 | 96.78 | 2.35 | 0.45 | 0.43 |
| | Ratio 0.04 | 96.90 | 2.08 | 0.53 | 0.49 |
| | Ratio 0.03 | 97.14 | 2.01 | 0.51 | 0.34 |
| | Ratio 0.02 | 96.25 | 2.05 | 1.22 | 0.48 |

As shown in Table 3, when the total unit ratio of the debranching enzyme to the diastatic enzyme changed from 1:0.05 to 1:0.03, the glucose yield increased. That is, the smaller the proportion of the diastatic enzyme, the higher the glucose yield.

COMPARATIVE EXAMPLE 1

### The Comparison of Amylopectin Degradation Activities of Isoamylases Derived from Various Strains (Activity Evaluation under the Glycosylation Conditions (60° C., pH 4.3))

TABLE 4

| | | | Activity |
|---|---|---|---|
| No. | Enzyme | Strain | (unit) |
| 1 | Isoamylase | Enterobacter cloacae subsp. Cloacae | 0 |
| 2 | | Bifidobacterium longum subsp. Longum | 0 |
| 3 | | Thermotoga neapolitana | 0 |
| 4 | | Escherichia coli str. K-12 substr. W3110 | 0 |
| 5 | | Sinorhizobium meliloti | 0 |
| 6 | | Acidothermus cellulolyticus | 130.18 |
| 7 | | Novosphingobium aromaticivorans | 0 |
| 8 | | Thermotoga thermarum | 0 |
| 9 | | Rhodothermus marinus | 105.04 |

The amylopectin degradation activities of isoamylases derived from various strains under the glycosylation conditions were compared. The results are shown in Table 4. Only the enzymes derived from Acidothermus cellulolyticus and Rhodothermus marinus were found to be active for the degradation of amylopectin. Thereafter, the yields of glucose after treatment with the two enzymes were compared.

### COMPARATIVE EXAMPLE 2

### The Comparison of Glucose Yields after Treatment with the Debranching Enzymes (Isoamylases) Derived from Acidothermus cellulolyticus and Rhodothermus marinus

The yields of glucose from starch after treatment with the debranching enzymes derived from Acidothermus cellulolyticus and Rhodothermus marinus were investigated. The enzymatic reaction conditions were as follows.

First, Multiple enzymatic reactions with liquefied starch (DE 9.5) were performed at a temperature of 60° C. for 72 h, wherein a diastatic enzyme (Glucoamylase, DuPont company) and each of the debranching enzymes (isoamylase, pullulanase) were used in total unit ratios of 1:2 to 1:60. The reactions were quenched by heating at 100° C. for 5 min. Then, the amounts of glucose produced were measured from the ratios between the areas of peaks corresponding to the enzymatic reaction products by HPLC. As a result, higher glucose yields were obtained when the debranching enzyme (isoamylase) derived from Acidothermus cellulolyticus was used (see the results in Table 6) than when the debranching enzyme derived from Rhodothermus marinus was used (see the results in Table 5). In addition, starch sugars, including di-(DP2) and higher saccharides, were analyzed. As a result, when the debranching enzyme (isoamylase) derived from Acidothermus cellulolyticus was used, the proportions of the unreacted products (DP2, DP4+) which did not degrade to glucose were reduced, indicating relatively high yields of glucose.

TABLE 5

| Diastatic enzyme | Debranching enzyme | | | | |
|---|---|---|---|---|---|
| (Glucoamylase, DuPont | (Isoamylase derived from | | | | |
| company) | Rhodothermus) | Glucose (%) | DP2 (%) | DP3 (%) | DP4+ (%) |
| Ratio 1 | Ratio 2 | 95.88 | 2.48 | 0.35 | 1.29 |
| | Ratio 10 | 96.26 | 2.31 | 0.37 | 1.06 |
| | Ratio 20 | 96.34 | 2.24 | 0.44 | 0.98 |
| | Ratio 60 | 96.61 | 2.08 | 0.56 | 0.75 |

TABLE 6

| Diastatic enzyme | Debranching enzyme | | | | |
|---|---|---|---|---|---|
| (Glucoamylase, DuPont | (Isoamylase derived from | | | | |
| company) | Acidothermus) | Glucose (%) | DP2 (%) | DP3 (%) | DP4+ (%) |
| Ratio 1 | Ratio 2 | 95.91 | 2.31 | 0.57 | 1.21 |
| | Ratio 10 | 96.71 | 2.15 | 0.60 | 0.54 |
| | Ratio 20 | 96.94 | 1.99 | 0.63 | 0.43 |
| | Ratio 60 | 97.07 | 1.90 | 0.63 | 0.40 |

As shown in Tables 5 and 6, the higher glucose yields and smaller proportions of the unreacted products (DP2 and DP4) with relatively high degrees of polymerization were obtained when the isoamylase derived from Acidothermus cellulolyticus was used than when the debranching enzyme derived from Rhodothermus marinus was used. That is, the application of the inventive isoamylase to enzymatic reactions leads to an increase in glucose yield.

1. A method for producing glucose from starch sugars, comprising: treating a substrate solution containing starch sugars with a debranching enzyme derived from a heat-resistant and acid-tolerant Acidothermus sp. and a diastatic enzyme.

2. The method according to claim 1, wherein the debranching enzyme derived from an Acidothermus sp. is produced by inserting a gene encoding the debranching enzyme into a vector to produce an expression vector, transforming the expression vector into cells, and mass-producing the transformed cells.

3. The method according to claim 2, wherein the vector is pET-28a(+).

4. The method according to claim 1, wherein the debranching enzyme derived from an Acidothermus sp. is an isoamylase.

5. The method according to claim 1, wherein the Acidothermus sp. is Acidothermus cellulolyticus.

6. The method according to claim 1, wherein the transformed cells are any one of E. coli, a microorganism belonging to the genus Bacillus, yeast, a microorganism belonging to the genus Corynebacterium and a plant.

7. The method according to claim 1, wherein the starch sugars are amylopectin-type starch.

8. The method according to claim 1, wherein the starch sugars are liquefied ones.

9. The method according to claim 1, wherein the starch sugars are derived from any one crop selected from corn, tapioca, potato, and rice.

10. The method according to claim 1, wherein the substrate solution containing starch sugars is treated with the debranching enzyme derived from an Acidothermus sp. and the diastatic enzyme at a pH of 2 to 6.

11. The method according to claim 1, wherein the substrate solution containing starch sugars is treated with the debranching enzyme derived from an Acidothermus sp. and the diastatic enzyme at a temperature of 40° C. to 70° C.

12. The method according to claim 1, wherein the total unit ratio of the debranching enzyme derived from an Acidothermus sp. to the diastatic enzyme is from 2:1 to 60:1.

13. A method of using a debranching enzyme derived from a heat-resistant and acid-tolerant Acidothermus sp. in the production of glucose from starch sugars.

14. A debranching enzyme derived from Acidothermus cellulolyticus that is active at a temperature of 40° C. to 70° C. and a pH of 2 to 6.
